(19) United States
(12) Patent Application Publication
(10) Pub. No.: US 2001/0006965 A1
(43) Pub. Date: Jul. 5, 2001

(54) Method for treating patients with diabetic retinopathy by administering substituted sulfonyl indenyl acetic acids and alcohols

(76) Inventors: Rifat Pamukcu, Spring House, PA (US); Gary Piazza, Doylestown, PA (US); Ewa Skopinska-Rozewska, Warsaw (PL)

Correspondence Address: Cell Pathways, Inc., 702 Electronic Drive, Horsham, PA, 19044, US

(21) Appl. No.: 09/779,103

(22) Filed: Feb. 8, 2001

Related U.S. Application Data
(63) Continuation of application No. 09/418,771, filed on Oct. 15, 1999, now abandoned.
Application No. 09/418,771 is a continuation of application No. 09/061,716, filed on Apr. 17, 1998, now abandoned.

(51) Int. Cl. A61K 31/535; A61K 31/19; A61K 31/18; A61K 31/165; A61K 31/05

(52) U.S. Cl. 514/239.5; 514/569; 514/602; 514/617; 514/732

(57) ABSTRACT

Substituted indenyl sulfonyl acetic acids, esters and alcohols are useful in the treatment of diabetic retinopathy.

### BACKGROUND OF THE INVENTION

This invention relates to therapeutic methods and pharmaceutical compositions for the treatment of diabetic retinopathy.

Diabetic retinopathy is a potentially blinding complication of diabetes that is characterized by temporary or permanent blindness. It affects half of the 14 million Americans with diabetes, even in pregnant women with pregnancy-induced diabetes. The condition often has no early warning signs and can be present even when patients notice no changes in their vision. But the disease is often progressive and can threaten vision. While timely treatment can ensure that 90 percent of those with advanced diabetic retinopathy will not become completely blind, many of those that do not go blind have visual impairments; and for those that go blind, there is no cure. Also, it can be difficult at the onset of the disease to ascertain which patients will suffer significant visual impairment or blindness from the disease.

Diabetic retinopathy affects the retina, which is the light-sensitive tissue at the back of the eye. When light enters the normal eye, the retina changes the light into nerve signals. The retina then sends these signals along the optic nerve to the brain. Without a functional retina, the eye cannot communicate with the brain, making vision impossible. Diabetic retinopathy causes damage to the tiny blood vessels in the retina that nourish it. Initially, most patients do not notice any changes in their vision. However, some people develop macular edemas that occur when the damaged blood vessels leak fluid and lipids onto the macula, the region of the retina with greatest visual sensitivity. The fluid makes the macula swell, resulting in blurred vision, for example, making reading and driving difficult. The disease can progress into a proliferative stage where fragile, new blood vessels grow along the retina and in the clear, gel-like vitreous that fills the inside of the eye. Without timely treatment, these new blood vessels can bleed, cloud vision, and destroy the retina.

As new blood vessels form at the back of the eye, they can bleed (hemorrhage) and blur vision. The first occurrence may not be very severe. In most cases, it will leave just a few specks of blood, or spots, floating in the patient's vision, which often goes away after a few hours. These spots are often followed within a few days or weeks by a much greater leakage of blood that will blur vision. In extreme cases, a person will only be able to tell light from dark in the afflicted eye. It may take the blood anywhere from a few days to months or even years to clear from inside of the eye. In some cases, the blood will not clear. Large hemorrhages unfortunately tend to occur more than once, often during sleep.

There are two current treatments for diabetic retinopathy: laser surgery and vitrectomy. Both of these therapies, however, are employed only when the disease is sufficiently advanced to warrant the attendant risks. In addition, both are primarily effective in reducing the amount of vision loss, not in eliminating the loss. Laser surgery is indicated for macular edema—often more than once to control the leaking fluid. During the surgery, high-energy beam of laser light is aimed directly onto the damaged blood vessels, which is called “focal laser treatment.” This seals the vessels and stops them from leaking. Generally, laser surgery is used to stabilize vision, not necessarily to improve it.

In the case of proliferative retinopathy, doctors use the laser to destroy the abnormal blood vessels that form at the back of the eye. Rather than focus the light on a single spot, hundreds of small laser bursts burn away from the center of the retina. This is called “scatter laser treatment,” which shrinks the abnormal blood vessels. Such treatment creates a loss of peripheral vision to save the more visually important central vision. Laser surgery may also reduce color and night vision.

A vitrectomy is indicated when the patient has a considerable amount blood in the vitreous. A vitrectomy involves removing the cloudy vitreous and replacing it with a salt solution.

Although laser surgery and vitrectomy can be “successful,” they do not cure diabetic retinopathy. A patient who has proliferative retinopathy is at risk for new bleeding. This means the patient may need treatment more than once to protect his sight.

There have been proposed pharmaceutical treatments for diabetic retinopathy, but none has been successful. In particular, the literature is replete with references to aldose reductase inhibitors as potential cures for diabetic retinopathy, but thus far, such compounds have not been successfully demonstrated to be effective in the clinic in afflicted patients.

### SUMMARY OF THE INVENTION

This invention relates to a method for treating diabetic retinopathy by administering to an afflicted patient a therapeutically effective amount of a compound of Formula I:

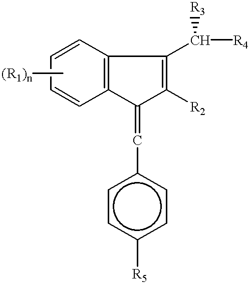

wherein R₁ is independently selected in each instance from the group consisting of hydrogen, halogen, lower alkoxy, hydroxy, lower alkyl, lower alkyl mercapto, lower alkylsulfonyl, lower alkylamino, di-lower alkyl amino, amino, nitro, nitrile, lower alkyl carboxylate, —CO₂H, and sulfonamido;

R₂ is selected from the group consisting of hydrogen and lower alkyl;

R₃ is selected from the group consisting of hydrogen, lower alkyl, hydroxy, and amino,

R₄ is selected from the group consisting of —COM and CH₂OH where M is selected from the group consisting of hydroxy, substituted lower alkoxy, amino, alkylamino, dialkylamino, N-morpholino, hydroxyalkylamino, polyhydroxyamino, dialkylaminoalkylamino, aminoalklyamino, and the group OMe, wherein Me is a cation;

R₅ is an alkyl sulfonyl; and n is an integer from 0 to four.

### DETAILED DESCRIPTION OF THE PREFERRED EMBODIMENTS

As explained above, this invention relates to a method of treating diabetic retinopathy by administering to an afflicted patient a therapeutically effective amount of a compound of Formula I above.

Preferred compounds within the scope of Formula I include those wherein R₁ is halogen; n is 1; R₂ is lower alkyl; M is hydroxy; and R₃ is hydrogen or lower alkyl. Most preferred compounds useful in therapeutic methods of this invention include those wherein R₁ is 5-fluoro; n is 1; R₂ is methyl; M is hydroxy; and R₃ is hydrogen.

The term “diabetic retinopathy” as used herein includes diabetic damage to the tiny blood vessels in the retina manifested in macular edema, proliferative retinopathy, and diabetes-induced damage to the vitreous.

As used herein, the term “alkyl” refers to straight, branched or cyclic alkyl groups and to substituted aryl alkyl groups. The term “lower alkyl” refers to C₁ to C₈ alkyl groups.

The term “lower alkoxy” refers to alkoxy groups having from 1 to 8 carbons, including straight, branched or cyclic arrangements.

The term “lower alkylmercapto” refers to a sulfide group that is substituted with a lower alkyl group; and the term “lower alkyl sulfonyl” refers to a sulfone group that is substituted with a lower alkyl group.

The term “lower alkyl carboxylate” refers to a carboxylate group that is substituted with a lower alkyl group.

The term “pharmaceutically acceptable salt” refers to non-toxic acid addition salts and alkaline earth metal salts of the compounds of Formula I. The salts can be prepared in situ during the final isolation and purification of such compounds, or separately by reacting the free base or acid functions with a suitable organic acid or base, for example. Representative acid addition salts include the hydrochloride, hydrobromide, sulfate, bisulfate, acetate, valerate, oleate, palmatate, stearate, laurate, borate, benzoate, lactate, phosphate, tosylate, mesylate, citrate, maleate, fumarate, succinate, tartrate, glucoheptonate, lactobionate, lauryl sulfate salts and the like. Representative alkali and alkaline earth metal salts include the sodium, calcium, potassium and magnesium salts.

It will be appreciated that certain compounds of Formula I can possess an asymmetric carbon atom and are thus capable of existing as enantiomers. Unless otherwise specified, this invention includes such enantiomers, including any racemates. The separate enaniomers may be synthesized from chiral starting materials, or the racemates can be resolved by conventional procedures that are well known in the art of chemistry such as chiral chromatography, fractional crystallization of diastereomeric salts and the like.

Compounds of Formula I also can exist as geometrical isomers (Z and E); the Z isomer is preferred.

Compounds useful in the practice of this invention may be formulated into pharmaceutical compositions together with pharmaceutically acceptable carriers for oral administration in solid or liquid form, or for rectal, intravenous, intramuscular, subcutaneous, transdermal, topical, opthalmic (topical or intraocular), although carriers for opthalmic administration are most preferred.

Pharmaceutically acceptable carriers for oral administration include capsules, tablets, pills, powders, troches and granules. In such solid dosage forms, the carrier can comprise at least one inert diluent such as sucrose, lactose or starch. Such carriers can also comprise, as is normal practice, additional substances other than diluents, e.g., lubricating agents such as magnesium stearate. In the case of capsules, tablets, troches and pills, the carriers may also comprise buffering agents. Carriers such as tablets, pills and granules can be prepared with enteric coatings on the surfaces of the tablets, pills or granules. Alternatively, the enterically coated compound can be pressed into a tablet, pill, or granule, and the tablet, pill or granules for administration to the patient. Preferred enteric coatings include those that dissolve or disintegrate at colonic pH such as shellac or Eudraget S.

Pharmaceutically acceptable carriers include liquid dosage forms for oral administration, e.g., pharmaceutically acceptable emulsions, solutions, suspensions, syrups and elixirs containing inert diluents commonly used in the art, such as water. Besides such inert diluents, compositions can also include adjuvants such as wetting agents, emulsifying and suspending agents, and sweetening, flavoring and perfuming agents.

Pharmaceutically acceptable carriers for topical administration include DMSO, alcohol or propylene glycol and the like that can be employed with patches or other liquid-retaining material to hold the medicament in place on the skin so that the medicament will not dry out.

Pharmaceutically acceptable carriers for rectal administration are preferably suppositories that may contain, in addition to the compounds of this invention excipients such as cocoa butter or a suppository wax, or gel. Pharmaceutically acceptable carriers for intraveneous administration include solutions containing pharmaceutically acceptable salts or sugars. Pharmaceutically acceptable carriers for intramuscular or subcutaneous injection include pharmaceutically acceptable salts, oils or sugars.

When used in its acid form, a compound useful in the practice of his invention can be employed in the form of a pharmaceutically acceptable salt of the acid. For example, sodium or potassium salts can be obtained by neutralizing with an equivalent base (alkali) metal hydroxide, mesylate of tosylate. When the active chemical is a base, it can be used as an acceptable formulation by neutralizing it with a suitable acid such as hydrochloric acid. Carriers such as solvents, water, buffers, alkanols, cyclodextrans and aralkanols can be used. Other auxiliary, non-toxic agents may be included, for example, polyethylene glycols, antimicrobial agents and wetting agents.

Ophthalmic suspensions for treating retinopathy in the mammalian, human and animal, eye using compounds of this invention can be prepared by employing ratios of the various proportional amounts of medicament and vehicle. Thus, the ophthalmic formulation can comprise from 0.001 to 10 mg/ml of the medicament. Examples of such formulations are provided below.

The ophthalmic preparation can contain certain excipients whose presence is desirable in preparing an acceptable ophthalmic preparation. The nature and proportional amounts of these excipients will be discussed in detail hereinafter. Thus in such a formulation, the objective is to administer the medicament at a desired dose from 0.001 to 10 mg/ml or an equivalent amount of the salt of such a medicament, and the remainder being inert carrier, excipient, preservative and the like.

An ophthalmic preparation can also be a suspension prepared with flocculating, deflocculating or suspending agents in various combinations and proportions. Known flocculating agents include alkanols of 1 to 4 carbon atoms, and aromatic alcohols sepected from the group consisting of benzyl alcohol, beta -phenyl-ethyl alcohol and cinnamyl alcohol, and mixtures of the above. Mixtures of varying proportions are suitable, and, for example, a mixture of benzyl alcohol and beta -phenylethyl alcohol.

The deflocculating or suspending agents employed in the ophthalmic suspension compositions are products derived from the condensation of polymers of ethylene oxide, and esters of fatty acids. Examples include from fatty acid esters of sorbitol, particularly the lauric, stearic and oleic acid esters of sorbitol. The fatty acid esters may be employed as mixtures from naturally occurring oils, which are esters of fatty acids and glycerol. Thus, the deflocculating agent may be polyoxyethylene. Naturally occurring fatty acid mixtures may be employed to produce esters of sorbitol for condensation with polyoxyethylene. Thus, the deflocculating agent may be polyoxyethylene sorbitol lanolin, polyoxyethylene sorbitol tallow esters, and polyoxyethylene sorbitol tall oil. Particularly preferred are esters of sorbitol and specific fat acids, especially lauric, stearic and oleic acids. Thus, the deflocculating agent may be polyoxyethylene sorbitan monolaurate, polyoxyethylene sorbitan monostearate, or polyoxyethylene sorbitan monoleate.

By use of the particular flocculating and deflocculating agents described above, it is possible to obtain acceptable ophthalmic suspension compositions which have the highly desirable properties of having the suspended material uniformly dispersed therein during the period of administration to the eye of the patient, while at the same time facilitating easy redispersion of that material after its flocculation and separation in the ophthalmic suspension composition.

In addition to the medicament, flocculating and deflocculating agents and water, conventional excipients and other materials are advantageously employed in preparing the ophthalmic suspension compositions of the present invention in accordance with good pharmaceutical practice. For example, the ophthalmic suspensions are sterile and preferably contain a bacteriological preservative to maintain sterility during use. Quaternary ammonium bacteriostats such as benzalkonium chloride may be used as well as phenyl mercuric acetate, phenyl mercuric nitrate, trimerosal, benzyl alcohol, or beta -phenylethyl alcohol. These bacteriostats may suitably be used in a range of from 0.01 to 3.0 mg./ml. and preferably 0.1 to 0.2 mg./ml. of total suspension. As antioxidant may also be used to prevent oxidation of the medicament. Suitable antioxidants include sodium bisulfate, N-acetyl cysteine salts, sodium ascorbate, sodium meta bisulfite, sodium acetone bisulfite and other acceptable anti-oxidants known to the pharmaceutical art. In conjunction with the antioxidants, chelating agents such as disodium edetate may also be employed.

Viscosity-inducing agents helpful in suspension characteristics of the composition, including cellulose derivatives such as hydroxymethyl cellulose, hydroxypropyl cellulose and methyl cellulose, may also be used in the formulation. Lecithin may also be used to produce helpful suspension characteristics for the ophthalmic suspension composition. A humectant is also sometimes used to help retain the water of the formulation in the eye. High molecular weight sugars are suitably used for this purpose such as sorbitol and dextrose in a suitable concentration.

The pharmaceutically acceptable carrier and compounds of this invention are formulated into unit dosage forms for administration to a patient. The dosage levels of active ingredient (i.e., compounds of this invention) in the unit dosage may be varied so as to obtain an amount of active ingredient effective to achieve activity in accordance with the desired method of administration (i.e., oral, rectal, or opthalmic suspension). The selected dosage level therefore depends upon the nature of the active compound administered, the route of administration, the desired duration of treatment, and other factors. If desired, the unit dosage may be such that the daily requirement for active compound is in one dose, or divided among multiple doses for administration, e.g., two to four times per day.

The pharmaceutical compositions of this invention are preferably packaged in a container (e.g., a box or bottle, or both) with suitable printed material (e.g., a package insert) containing indications, directions for use, etc.

Examples 1-3 illustrate compounds useful in the practice of the claimed invention.

### EXAMPLE 1

### α-(1-p-Methylsulfonylbenzylidene)-2-Methyl-5-Fluoro-3-Indeny-1-Acetic Acid

(A) p -Fluoro-α-methylcinnamic acid

p-Fluorobenzaldehyde (200 g., 1.61 mole), propionic anhydride (3.5 g., 2.42 mole) and sodium propionate (155 g., 1.61 mole) are mixed in a 1 liter, three-necked flask flushed with nitrogen. The mixture is heated gradually in an oil-bath to 140° C. After 20 hours, the flask is cooled to 100° C. and poured into 8 1. of water. The precipitate is dissolved by adding potassium hydroxide (302 g) in 2.1 of water. The aqueous solution is extracted with ether, and the ether extracts are washed with potassium hydroxide solution. The combined aqueous layers are filtered, acidified with concentrated HCl, and filtered; and the collected solid washed with water, thereby producing p-fluoro-α-methylcinnamic acid which is used as obtained.

(B) p-Fluoro-α-methylhydrocinnamic acid

To p-fluoro-α-methylcinnamic acid (177.9 g., 0.987 mole) in 3.6 l. ethanol is added 11.0 g. of 5% Pd/C and the mixture is reduced at room temperature under a hydrogen pressure of 40 p.s.i. uptake is 31/32 lbs. (97% of theoretical). After the catalyst is filtered, the filtrate is concentrated in vacuo to give the product, p -fluoro-α-methylhydrocinnamic acid that is used without weighing in next step.

(C) 6-Fluoro-2-methylindanone

To polyphosphoric acid (932 g) at 70° C. on the steam bath is added p-fluoro-α-methylhydrocinnamic acid (93.2 g., 0.5 mole) slowly with stirring. The temperature is gradually raised to 95° C., and the mixture is kept at that temperature for 1 hour. The mixture is allowed to cool and added to 2 l. of water. The aqueous layer is extracted with ether, the ether solution washed twice with saturated sodium chloride solution, 5% Na₂CO₃ solution, water, and then dried. The ether filtrate is concentrated with 200 g. silica-gel, and added to a five pound silica-gel column packed with 5% ether-petroleum ether. The column is eluted with 5-10% ether-petroleum ether and followed by TLC to give 6-fluoro-2-methylindanone.

(D) 5-fluoro-2-methylindanone-3-acetic acid

A mixture of 6-fluoro-2-methylindanone (18.4 g., 0.112 g. mole), cyanoacetic acid (10.5 g., 0.123 mole), acetic acid (6.6 g.), and ammonium acetate (1.7 g.) in dry toluene (15.5 ml.) is refluxed with stirring for 21 hours, as the liberated water is collected in a Dean Stark trap. The toluene is concentrated, and the residue dissolved in 60 ml of hot ethanol and 14 ml. of 2.2N aqueous potassium hydroxide solution. 22 g. of 8.5% KOH in 150 ml of water is added, and the mixture refluxed for 13 hours under nitrogen. The ethanol is removed under vacuum, water (500 ml) is added; and the aqueous solution washed well with ether and then boiled with charcoal. The aqueous filtrate is acidified to pH 2 with 50% hydrochloric acid, cooled and the precipitate collected. In this way dried 5-fluoro-2-methylindenyl-3-acetic acid (M.P. 164-166° C.) is obtained.

(E) 5-fluoro-2-methyl-1-(p-methylthiobenzylidene)-3-indenyl acetic acid

5-fluoro-2-methyl-3-indenyl acetic acid (15 g., 0.072 mole) p-methylthiobenzaldehyde (14.0 g., 0.091 mole) and sodium methoxide (13.0 g., 0.24 mole) are heated in methanol (200 ml.) at 60 degree(s) under nitrogen with stirring for 6 hours. After cooling, the reaction mixture is poured into ice-water (750 ml), acidified with 2.5N hydrochloric acid, and the collected solid triturated with a little ether to produce 5-fluoro-2-methyl-1-(p-methylthiobenzylidene)-3-indenyl acetic acid (M.P. 187-188.2° C.). U.V. in methanol λmax. 348 mμ (E % 500), 258 (557), 258 (495), 353 (513), 262.5 (577), 242.5. (511).

(F) 5-fluoro-2-methyl-1-(p-methylsulfinylbenzylidene)-3-indenyl acetic acid

To a solution of 5-fluoro-2-methyl-1-(p-methylthiobenzylidene)-3-indenyl acetic acid (3.4 g., 0.01 mole) in a mixture of methanol (250 ml.) and acetone (100 ml.) is added a solution of sodium periodate (3.8 g., 0.018 mole) in water (50 ml.) with stirring. Water (450 ml.) is added after 18 hours, and the organic solvents removed under vacuum below 30° C. The precipitated product is filtered, dried and recrystallized from ethyl acetate to give 5-fluoro-2-methyl-1-(rho-methylsulfinylbenzylidene)-3-indenyl acetic acid. Upon repeated recrystallization upon ethylacetate there is obtained cis-5-fluoro-2-methyl-1-(p-methylsulfinylbenzylidene)-3-indenyl acetic acid, M.P. 184-186° C. U.V. in methanol; λmax 328 (E % 377), 286 (432), 257.5 shldr. (413), 227 (548). Further runs reveal the existence of a second polymorph of cis-5-fluoro-2-methyl-1-(p-methylsulfinylbenzylidene)-3-indenyl acetic acid, M.P. 179-181° C. 5—Chloro-2-methyl-1-(p-methylsulfinylbenzylidene)-3-indenyl acetic acid is prepared by the procedure as described previously in this Example, and can be converted to the corresponding sulfonyl compound by the procedure set forth below.

5-fluoro-2-methyl-1-(p-methylsulfonylbenzylidene)-3 -indenyl acetic acid is prepared by adding sodium methoxide (4.4M in MeOH, 68.5 ml, 0.3 mol) dropwise to a stirred, cooled mixture of 5-fluoro-2-methyl-1-(p-methylsulfinylbenzylidene)-3-indenyl acetic acid (100 g, 0.281 mol) in methanol (250 ml) and acetonitrile (500 ml). Sodium bicarbonate (0.56 mol) and hydrogen peroxide (30% in water, 0.56 mol) are added and allowed to react for 18 hours at −10° C. Excess sodium bicarbonate is filtered off, and cooled filtrate (0° C.) neutralized dropwise to pH 7 with 1M hydrochloric acid (350 ml). The resulting product is then filtered and washed with methanol. A thin layer chromatography system to check for purity utilizes chloroform:methyl isobutyl ketone (8:2); the R_{f} value is 0.21. A tetrahydrofuran/diisopropyl ether combination can be used for product recrystallization. Reaction yield is 89%. (R₁═5-fluoro; R₂═CH₃; R₃═hydrogen; R₄═COOH; R₅═CH₃ SO₂; n=1).

Formula: C₂₀H₁₇FO₄S

Molecular Mass: 372.41 g/mol

Melting point: 204-206° C.

¹H-NMR [ppm] DMSO-d₆): 2.16 (s, 3, —CH₃); 3.30 (s, 3, —SO₂—CH₃); 3.59 (s, 2, —CH₂—C═O);

6.70-7.17 (m, 3, ar.); 7.38 (s, 1, ═CH—); 7.78-8.04 (AB, 4,, —PH—SO₂—);

HPLC (C-18 Column, 50% acetic acid (2%)/50% acetonitrile, 1.5 ml/min):

IR [cm⁻¹] (KBr): 1710 C═O; 1310 S═O; 1180 C—F; 1140 S═O;

α-[1-(p-Methylsulfonylbenzylidene)-2-methyl-5-fluoro-3-indenyl]-propionic acid is prepared by the similar procedures known in the art.

### EXAMPLE 2

### α-(1-p-Methylsulfonylbenzylidene)-2-Methyl-5-Fluoro-3-Indeny-1-Acetic Acid Methyl Ester

5-Fluoro-2-methyl-1-(p-methylsulfonylbenzylidene)-3-indenyl acetic acid is prepared by the procedure of Example 1, and converted to the methyl ester derivative by the following procedure. Sodium methoxide (4.4M in methanol, 1.36 ml, 0.006 mol) is added to a stirred cooled solution (0 degree(s) C.) of 5-fluoro-2-methyl-1-(p-methylsulfonylbenzylidene)-3-indenyl acetic acid (1.04 g, 0.0028 mol) in methanol (5 ml) and acetonitrile (10 ml). After 30 minutes, the reaction mixture is dropped into concentrated hydrochloric acid (50 ml) and extracted with methylene chloride (3×25 ml). The organic layer is extracted with saturated sodium bicarbonate (3×25 ml), dried with sodium sulfate, and concentrated in vacuo. The resulting oil is crystallized from tetrahydrofuran/hexane to yield 0.2 g of the desired compound. The melting point is 165-166° C. (R₁=5-fluoro; R₂═CH₃; R₃═hydrogen; R₄═COO CH₃; R₅═CH₃ SO₂; n=1). Other methyl esters of compounds useful in this invention can be prepared in a similar fashion.

### EXAMPLE 3

### (Z)-5-Fluoro-2-Methyl-1-(4-Methylsulfonylbenzylidene)-1H-3-Indenyl-(2-Hydroxy) Ethane

(A) Methyl-5-fluoro-2-methyl-1H-3-indenylacetate

Nitrosomethylurea (99.5 mmol) is added in portions to a cold (0° C.) mixture of aqueous 50% KOH (50 ml) and diethylether (150 ml) at 0° C. The yellow ether solution of diazomethane (Note: explosive) is separated, is washed with water, and is added in portions to a solution of 5-fluoro-2-methylindene-3-acetic acid (90 mmol) in dichloromethane (200 ml). When the evolution of N₂ ceases, the reaction is complete. After evaporation of the solvents, the residue is recrystallized from hexane to give methyl 5-fluoro-2-methyl-3-indenylacetate (yield 93%; m.p. 53° C.).

(B) 5-Fluoro-2-methyl-1H-3-indenyl-(2-hydroxy) ethane

To a solution of methyl 5-fluoro-2-methyl-3-indenyl-acetate (24 g) in dry THF (300 ml) lithiumaluminum hydride (6.9 g) is added. The mixture is stirred at room temperature for 1.5 hours. Excess LiA1H4 is destroyed with saturated aqueous NaHSO₄ solution. The organic phase is concentrated in vacuo, and the crude product is purified via silica gel column chromatography elution with methylene chloride. The residue is recrystallized from hexane to give 5-fluoro-2-methyl-1H-3-indenyl-(2-hydroxy) ethane (yield 63%; m.p. 65°-66.5° C.).

(C) (Z)-5-Fluoro-2-Methyl-1-(4-Methylsulfonylbenzylidene)-1H-3-Indenyl-(2-Hydroxy) Ethane

5-Fluoro-2-methyl-1H-3-indenyl-(2-hydroxy) ethane (15 g, 0.072 mol) p-methylsulfonylbenzaldehyde (14.0 g, 0.091 mol) and sodium methoxide (13.0 g, 0.24 mol) are heated in methanol (200 ml) at 60° C. under nitrogen with stirring for 6 hours. The reaction mixture is poured onto ice-water (750 g), and is acidified with 2.5N hydrochloric acid. The collected solid is triturated with a little ether to produce (Z)-5-fluoro-2-methyl-1-(p-methylsulfonylbenzylidene)-1H-3-indenyl-(2-hydroxy) ethane. Recrystallization of the crude reaction product results in the separation of the mixture of geometrical isomers (Z/E) and gives the title compound (R₁=5-fluror, R₂═CH₃R₃═H, R₄═CH₂OH, n=1, R₅═CH₃ SO₂).

Formula: C₂₀H₁₀FO₃S

Molecular Mass: 358.43 g/mol

Melting point: 118° C.

¹H-HMR [ppm] (DMSO-d₆): 2.14 (s, 3, —CH₃); 2.71 (t, 2, —CH₂—); 3.29 (s, 3, —SO₂—CH₃); 3.55 (M, 3, —CH₂—O); 4.70 (m, 1, —OH); 6.68-7.14 (m, 3, ar.); 7.30 (s, 1, ═CH); 7.76-8.03 (AB, 4, —Ph—SO₂—);

IR [cm⁻¹] (KBr): 3440 OH; 1300 S═O; 1170 C═F; 1140 S═O

The remaining examples illustrate preparation of ophthalmic suspensions incorporating the compounds above.

### EXAMPLES 4-6

The following materials are admixed in a 1250 ml. bottle: 20.6 g. of 5-fluoro-2-methyl-1-(p-methylsulfonylbenzylidene)-3-indenyl acetic acid (Example 1) which is a sufficient amount of medicament to result in a concentration of approximately 10 mg. per ml. in the final samples, allowing for previously established 3.0% average; 0.4 g. sodium bisulfite, 12 g. NaCl, and 28 ml. water (at 180 degree(s) F.) The mixture (IV) is autoclaved for 30 minutes at 121 degree(s) C. under 15 psig. Separately, 3 g. of hydroxyethylcellulose in 720 ml. of water (V) and 0.4 g. of lecithin in 80 ml. of water (VI) are autoclaved for 30 minutes at 121 degree(s) C. Then, VI is admixed with I for 2 hours, and the resulant mixture is poured into II. Another mixture (VII) is prepared from 20 g. of sorbitol, 2.36 ml. of benzalkonium chloride, 10 g. of disodium edetate, and water to give a final solution volume of 900 ml. Then, VII is added to the mixtures of IV, V, and VI in sufficient quantity to give 1.8 l. overall. The 1.81 mixture of IV, V, VI and VII is then taken and homogenized using a homogenizer at 2000 psig. Stock solutions are then prepared for polyoxyethylene (20) sorbitan monooleate by dissolving 3 g. of the material in 100 ml. of water, and of benzyl alcohol/beta -phenylethyl alcohol by admixing 50 ml. of each alcohol. Varying quantities of the two stock solutions are then added to four 90 ml. aliquots of the homogenized mixture of IV, V, VI and VII prepared as described above, together with sufficient water to give a total of 100 ml. for each of four different samples.

### EXAMPLE 7

### Solution Composition

(Z)-5-Fluoro-2-Methyl-1-(4-Methylsulfonylbenzylidene)-1H-3-Indenyl-(2-Hydroxy) Ethane (Example 2) (0.1 mg.) and peanut oil (0.10 mg) are mixed together. The solution is rendered sterile by filtration through a sterilizing filter.

### EXAMPLE 8

(Z)-5-fluoro-2-methyl- 1-(p-methylsulfonylbenzylidene)-1H-3 -indenyl-(2-hydroxy) ethane (0.5 mg) and petrolatum (I grain) are aseptically combined.

EXAMPLE 9
Topical Ocular Suspension

| | Amount (wt %) |
|---|---|
| Compound of Example 1 | 0.001-1 |
| HPMC | 0.5 |
| Sodium chloride | 0.8-0.9 |
| Benzalkonium chloride | 0.01 |
| EDTA sodium | 0.01 |
| Sodium hydroxide/sodium bicarbonate | qs pH 7.4 |
| Distilled water | qs 100 ml |

EXAMPLE 10
Formulation for Sterile Intraocular or Intravenous Injection

| | Amount |
|---|---|
| | (grams/Liter) |
| Compound of Example 1 | 0.01-10 |
| Sodium chloride | 13.04 |
| Potassium chloride | 0.712 |
| Sodium chloride (monobasic) | 0.206 |
| Sodium bicarbonate | 4.908 |
| Calcium chloride | 0.306 |
| Magnesium chloride | 0.318 |
| Dextrose | 1.8 |
| NaOH/HCl | Qs to pH 7.4 |
| Hydroxypropylcyclodextran | 0.1-1% (as a carrier) |

It will be understood that various changes and modifications may be made in the details of procedure, formulation and use without departing from the spirit of the invention, especially as defined in the following claims.

We claim:

1. A method for treating a patient with diabetic retinopathy sensitive to compounds below, comprising administering to the patient a physiologically effective amount of a compound of the formula: 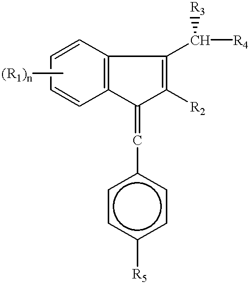
wherein R₁ is independently selected in each instance from the group consisting of hydrogen, halogen, lower alkoxy, hydroxy, lower alkyl, lower alkyl mercapto, lower alkylsulfonyl, lower alkylamino, di-lower alkyl amino, amino, nitro, nitrile, lower alkyl carboxylate, —CO₂H, and sulfonamido;
R₂ is selected from the group consisting of hydrogen and lower alkyl;
R₃ is selected from the group consisting of hydrogen, lower alkyl, hydroxy, and amino;
R₄ is selected from the group consisting of —COM and CH₂OH wherein M is selected from the group consisting of hydroxy, substituted lower alkoxy, amino, alkylamino, dialkylamino, N-morpholino, hydroxyalkylamino, polyhydroxyamino, dialkylaminoalkylamino, aminoalklyamino, and the group OMe, wherein Me is a cation;
R₅ is an alkyl sulfonyl; and n is an integer from 0 to four.

2. The method of claim 1 wherein R₁ is halogen and n is 1.

3. The method of claim 2 wherein R₁ is 5-fluoro.

4. The method of claim 2 wherein R₂ is lower alkyl.

5. The method of claim 4 wherein R₂ is lower alkyl.

6. The method of claim 4 wherein M is hydroxy; and R₃ is selected from the group consisting of hydrogen or lower alkyl.

7. The method of claim 6 wherein R₃ is hydrogen.

8. An ophthalmic preparation comprising an opthalmically acceptable liquid carrier and a compound of the formula: 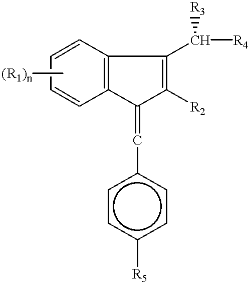
wherein R₁ is independently selected in each instance from the group consisting of hydrogen, halogen, lower alkoxy, hydroxy, lower alkyl, lower alkyl mercapto, lower alkylsulfonyl, lower alkylamino, di-lower alkyl amino, amino, nitro, nitrile, lower alkyl carboxylate, —CO₂H; and sulfonamido;
R₂ is selected from the group consisting of hydrogen and lower alkyl;
R₃ is selected from the group consisting of hydrogen, lower alkyl, hydroxy, and amino;
R₄ is selected from the group consisting of —COM and CH₂OH wherein M is selected from the group consisting of hydroxy, substituted lower alkoxy, amino, alkylamino, dialkylamino, N-morpholino, hydroxyalkylamino, polyhydroxyamino, dialkylaminoalkylamino, aminoalklyamino, and the group OMe, wherein Me is a cation;
R₅ is an alkyl sulfonyl; and n is an integer from 0 to four.
